SEQUENCE LISTING (22 sequences)

SEQ ID NO: 1 - DNA, 20 bp, Artificial sequence
  primer
agtccccatggagtcaaaga

SEQ ID NO: 2 - DNA, 20 bp, Artificial sequence
  primer
ctcaaggagacctttgctca

SEQ ID NO: 3 - DNA, 20 bp, Artificial sequence
  primer
tgtctctccagtttccctgc

SEQ ID NO: 4 - DNA, 20 bp, Artificial sequence
  primer
ttcagggactcatgggctca

SEQ ID NO: 5 - DNA, 20 bp, Artificial sequence
  primer
atgcctgtaatcccagcact

SEQ ID NO: 6 - DNA, 20 bp, Artificial sequence
  primer
tcatggtggtagctggtagc

SEQ ID NO: 7 - DNA, 20 bp, Artificial sequence
  primer
cacaggactagaacacctgc

SEQ ID NO: 8 - DNA, 19 bp, Artificial sequence
  primer
gctggtgaaaaggacctct

SEQ ID NO: 9 - DNA, 20 bp, Artificial sequence
  primer
ggatggcaatcgaatcactg

SEQ ID NO: 10 - DNA, 20 bp, Artificial sequence
  primer
tctgctctaggctgcttctt

SEQ ID NO: 11 - DNA, 21 bp, Artificial sequence
  primer
tgagccaagtgttaagtgtgg

SEQ ID NO: 12 - DNA, 20 bp, Artificial sequence
  primer
gagcaagctgcatcatttcc

SEQ ID NO: 13 - DNA, 22 bp, Artificial sequence
  primer
aggggaccagctgcacattagg

SEQ ID NO: 14 - DNA, 22 bp, Artificial sequence
  primer
aggccgcttctcttgaccactt

SEQ ID NO: 15 - DNA, 18 bp, Artificial sequence
  primer
gccagggggtctagaagc

SEQ ID NO: 16 - DNA, 20 bp, Artificial sequence
  primer
tcacttggcacccagtacaa

SEQ ID NO: 17 - DNA, 24 bp, Artificial sequence
  primer
ctcagagcttcggcgcatcaccag

SEQ ID NO: 18 - DNA, 24 bp, Artificial sequence
  primer
gacaaacaccacatccagaaccat

SEQ ID NO: 19 - DNA, 20 bp, Artificial sequence
  primer
aaccacttcacagagctgca

SEQ ID NO: 20 - DNA, 20 bp, Artificial sequence
  primer
caagccatcagcttctccat

SEQ ID NO: 21 - DNA, 22 bp, Artificial sequence
  primer
gactatggcagcagtctcttcc

SEQ ID NO: 22 - DNA, 21 bp, Artificial sequence
  primer
ggtggttgtcgtctgacaatt

(19) United States
(12) Patent Application Publication
(10) Pub. No.: US 2005/0208025 A1
(43) Pub. Date: Sep. 22, 2005

(54) Enhancement of hematopoietic stem cell survival

(76) Inventors: William H Fleming, Portland, OR (US); Bei Li, Lake Oswego, OR (US)

Correspondence Address: KLARQUIST SPARKMAN, LLP, 121 SW SALMON STREET, SUITE 1600, PORTLAND, OR, 97204, US

(21) Appl. No.: 10/511,233

(22) PCT Filed: Apr. 15, 2003
(86) PCT No.: PCT/US03/11649

§ 371 Date: Oct. 12, 2004

Related U.S. Application Data
(60) Provisional application No. 60/373,127, filed on Apr. 16, 2002.

(51) Int. Cl. A61K 48/00; C12N 5/08

(52) U.S. Cl. 424/93.7; 435/372

(57) ABSTRACT

A composition is disclosed that includes lin⁻ cells that are characterized as expressing CD31, CD34 and CD105, and not expressing c-kit, wherein the composition comprises fewer than 20% of lineage committed cells. Methods are disclosed for isolating CD31⁺CD34⁺CD45⁻CD105⁺c-kit⁻ cells. Methods for reconstituting hematopoiesis using compositions including CD31⁺CD34⁺CD105⁺CD45⁻kit⁻ cells are also disclosed.

### PRIORITY

This application claims the benefit of U.S. Provisional Application No. 60/373,127, filed Apr. 16, 2002, which is incorporated by reference in its entirety.

### STATEMENT OF GOVERNMENT SUPPORT

This invention was made with United States government support, pursuant to grants R01 (H1069133) and R01 (HD039251), from the National Institutes of Health. This invention was also made with support from grant RPG LBC-100677, awarded by the American Cancer Society. The United States government has certain rights in the invention.

### FIELD

This application relates to the field of hematopoietic stem cells, more specifically to cells and factors that promote the survival, proliferation, and/or differentiation of hematopoietic stem cells.

### BACKGROUND

Mammalian blood cells provide for an extraordinarily diverse range of activities. The blood cells are divided into several lineages, including lymphoid, myeloid and erythroid. The lymphoid lineage, comprising B cells and T cells, provides for the production of antibodies, regulation of the cellular immune system, detection of foreign agents in the blood, detection of cells foreign to the host, and the like. The myeloid lineage, which includes monocytes, granulocytes, megakaryocytes as well as other cells, monitors for the presence of foreign bodies in the blood stream, provides protection against neoplastic cells, scavenges foreign materials in the blood stream, produces platelets, and the like. The erythroid lineage provides the red blood cells, which act as oxygen carriers. Despite the diversity of the nature, morphology, characteristics and function of the blood cells, there appears to be a single progenitor, which is capable of self regeneration and by exposure to growth factors becomes dedicated to a specific lineage.

Recently, the mouse stem cell has been obtained in at least highly concentrated, if not a purified form, where fewer than about 30 cells obtained from bone marrow were able to reconstitute all of the lineages of the hematopoietic system of a lethally irradiated mouse. Indeed, one injected cell should be able to reconstitute all of the hematopoietic lineages. A human stem cell has also been identified that is a CD34⁺Thy1⁺lin⁻ cell. This stem cell population constitutes only a small percentage of the total number of leukocytes in bone marrow. In particular, B cells (CD19⁺) and myeloid cells (CD33⁺) make up 80-90% of the CD34⁺ population. Moreover, a combination of CD3, 8, 10, 15, 19, 20, and 33 will mark >90% of all CD34⁺ cells.

There is a strong interest in identifying factors and cells that promote the growth or differentiation of hematopoietic stem cells. The availability of cells that promote the growth or differentiation of stem cells would be extremely useful in bone marrow transplantation, as well as transplantation of other organs in association with the transplantation of bone marrow. Stem cells are also important targets for gene therapy, where the inserted genes promote the health of the individual into whom the stem cells are transplanted. In addition, the ability to isolate the cells and factors that interact with stem cells may serve in the treatment of lymphomas and leukemias, as well as other neoplastic conditions, e.g., breast cancer.

### SUMMARY

Cells are disclosed herein that are of vascular origin and express CD31. These cells can be used to reconstitute hematopoiesis in a subject. Methods of isolating cells of vascular origin that express CD31 are also disclosed.

A composition is disclosed herein that includes lin⁻ cells that are characterized as expressing CD31, CD34 and CD105, and not expressing c-kit. The composition comprises fewer than 20% of lineage committed cells. In one specific, non-limiting example the composition includes the composition comprises greater than about 80% CD31⁺34⁺CD45⁻CD105⁺ c-kit⁻lin⁻ cells. A composition is disclosed herein that includes substantially purified CD31⁺34⁺CD45⁻CD105⁺ c-kit⁻lin⁻ cells. Also described herein is an isolated cell that promotes hematopoietic stem cell survival, wherein the cell expresses CD31, CD34 and CD105, but does not express c-kit or a hematopoietic lineage specific marker.

In another embodiment, a method is disclosed for preparing a composition comprising a purified population of cells, wherein greater than 50% of the cells are CD31⁺CD34⁺CD45⁻CD105⁺c-kit⁻lin⁻ cells. The method includes contacting cells of the vasculature with an antibody that specifically binds CD31 and separating cells that bind the antibody from the vasculature to isolate the population of cells that are CD31⁺CD34⁺CD45⁻CD105⁺c-kit⁻lin⁻.

In yet another embodiment, a method is also disclosed for reconstituting hematopoiesis in a subject. The method includes administering to the subject a therapeutically effective amount of a composition comprising CD31⁺CD34⁺CD45⁻CD105⁺c-kit⁻lin⁻ cells, thereby reconstituting hematopoiesis.

In an additional embodiment, a method is disclosed for promoting proliferation or differentiation of a hematopoietic stem cell. The method includes co-culturing the hematopoietic stem cell with a CD31⁺CD34⁺CD45⁻CD105⁺lin⁻ c-kit⁻ cell.

The foregoing and other features and advantages will become more apparent from the following detailed description of several embodiments, which proceeds with reference to the accompanying figures.

### BRIEF DESCRIPTION OF THE FIGURES

FIG. 1 is a graph demonstration that CD31⁺ vascular endothelial cells provide protection from lethal irradiation. CD31⁺ cells were isolated from both the brain and lung and then purified by FACS cell sorting. FIG. 1A is a graph of survival of transplanted animals when lethally irradiated recipient mice were injected with brain derived CD31⁺ cells at a dose of 30 K cells per recipient (●), 10 K cells per recipient (□), 1 K cells per recipient (▪) or were injected with a vehicle control (∘). FIG. 1 B is a graph of survival when lung derived CD31⁺ cells were injected at doses of 10 K cells per recipient (□), or 1 K cells per recipient (▪) or with a vehicle control (∘). Survival was monitored daily. Combined results from at least two independent experiments is shown (n=10-14 recipients per treatment group).

FIG. 2 is a set of FACS plots documenting endothelial cell marker expression on CD31⁺ cells. Endothelial cells isolated from brain and lung and labeled with antibodies to CD31, CD34, Sca-1 and c-kit. The expression of these cell surface markers was evaluated by flow cytometry. The percentage of CD31⁺ cells expressing a specific cell surface marker is indicated (Sensitivity is ≧1.0%). Representative data from 4 independent experiments is shown.

FIG. 3 is a bar graph of the expression of endothelial lineage and hematopoietic lineage cell markers on CD31⁺ cells. Vascular endothelial cells (CD31⁺) were isolated from brain and lung tissue then labeled with lineage specific antibodies. Gated populations of CD31⁺ cells were evaluated for the expression of lineage specific markers by flow cytometry. (Sensitivity is ≧1.0% of gated CD31⁺ cells). Combined results from 4 independent experiments are shown.

FIG. 4 is a set of digital images showing gene expression profiles of brain and lung derived CD31⁺ cells. Vascular endothelial cells were isolated from the brain (FIG. 4A) or lung (FIG. 4B) and the CD31⁺ cells were purified by cell sorting. Total RNA was extracted from 2×10⁴ CD31⁺ cells or unfractionated normal bone marrow cells and analyzed for the expression of hematopoietic cell genes and endothelial cell genes by RT PCR. Normal unfractionated bone marrow was used as a positive control (FIG. 4C). The size of the expected PCR product is indicated. Representative and data from 3 independent experiments is shown.

FIG. 5 is a set of bar graphs showing long term multilineage hematopoiesis in recipients of CD31⁺ cells. FIG. 5A is a bar graph of the results obtained six months after transplantation, peripheral blood (PB) was obtained from the recipients radioprotected by brain derived CD31 cells or by a mixture of unfractionated bone marrow (BM) cells from donor mice that differ at the Ly5 locus. Nucleated peripheral blood cells were analyzed for the presence of with Ly5.2 donor derived cells and Ly5.1⁺ host derived cells in CD31⁺ cells recipients and BM♦. The mean percentage of donor derived and host derived cells in the peripheral blood is shown. (±SEM, * below the level of detection or <0.2%) (♦ Competitive repopulation assay of 5×10⁵ Ly5.1 donor cells and 1×10⁵ Ly5.1 host type cells). FIG. 5B is a bar graph showing multilineage hematopoietic reconstitution of brain derived CD31⁺ cell and BM recipients analyzed 6 months after transplantation. The frequency of total nucleated cells in the peripheral blood that express markers specific for B cells (B220), T cells (CD3), and myelomonocytic cell (Mac-1/Gr-1) is shown. The mean and SEM for each group is indicated.

FIG. 6 is a bar graph obtained after secondary transplantion. Eight months after primary transplant, bone marrow was obtained from the mice that had been radioprotected by 10 k CD31⁺ cells from brain, and transplanted into each secondary recipients, which were irradiated to a lethal dose of 1150 cGy. Each secondary recipients (n=5) received 2×10⁶ total BM cells from brain CD31 transplanted primary recipients. Multilineage reconstitution of peripheral blood from each recipient was analyzed at the time point of 6 weeks after secondary transplantation, and gated populations of both donor and host cells were analyzed for expression of markers of B-cells (B220), T-cells (CD3) and myelomonocytic cells (Mac-1/Gr-1). There was an average of 42.7% of primary recipient derived cells (□) which contains and 57.3% of secondary recipient type cells (▪).

### DETAILED DESCRIPTION

Terms

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of the invention, the following explanations of specific terms are provided:

Agent that affects hematopoiesis: A compound, antibody, cell, nucleic acid molecule, steroid or protein that affects hematopoiesis. In one embodiment, the agent affects the growth, proliferation, maturation, or differentiation of hematopoietic cells. An agent can be a naturally occurring molecule or a synthetic molecule. In one specific, non-limiting example, the agent is a pharmaceutical compound. In another specific non-limiting example, the agent is a protein, such as a growth factor. In yet another specific non-limiting example, the agent is a cell, such as a CD31⁺CD34⁺CD45⁻CD105⁺lin⁻c-kit⁻ cell.

Animal: Living multicellular vertebrate organisms, a category which includes, for example, mammals and birds.

B Cell: A B cell is a lymphocyte, a type of white blood cell (leukocyte), that develops into a plasma cell, which produces antibodies.

βH-1: The β chain of the hemoglobin molecule. The expression of PH-1 is restricted to cells committed to the erythroid cell lineage.

CD31: A 130 to 140-kdalton single-chain integral membrane glycoprotein that is a member of the immunoglobulin gene superfamily, and is also known as PE-cell adhesion molecule (CAM). The CD31 antigen is composed of six extracellular immunoglobulin-like domains belonging to the C2 group. C2 domains are also found in other members of the immunoglobulin superfamily, the CAMs. The CD31 antigen is expressed on endothelial cells and platelets, T lymphocyte subsets, monocytes, and granulocytes, and is known to function as a vascular cell adhesion molecule and is involved in the process of leukocyte migration through the intercellular junctions of vascular endothelial cells (see Stockinger et al., J Immunol. 145(11):3889-3897, 1990).

CD34: A cell surface antigen formerly known as hematopoietic progenitor cell antigen 1, and MY10, is a known marker of human hematopoietic stem cells. The human CD34 gene, which maps to chromosome 1q32, spans 26 kb and has 8 exons. CD34 is a 67 kDa transmembrane glycoprotein. CD34 is expressed selectively on human hematopoietic progenitor cells. The biological function of CD34 is still unknown.

C-kit: A proto-oncogene that encodes a transmembrane receptor with intrinsic tyrosine-specific protein kinase activity in its intracellular domain. It is the cellular homologue of the viral kit oncogene of HZ4-FSV (Hardy-Zuckerman 4 feline sarcoma virus). The kit receptor is also known as CD117. The ligand for the kit receptor is Stem Cell Factor.

Human c-kit maps to chromosome 4q11-q12 in the same region also encoding one of the platelet derived growth factor (PDGF) receptors (PDGFRA). The two genes have been located on a DNA fragment of approximately 700 kb. The human kit gene has a length of more than 70 kb and contains 21 exons. The longest transcript is 5230 bp and is alternatively spliced. A c-kit⁻ cell is a cell that does not express c-kit.

Cytokine: Proteins made by cells that affect the behavior of other cells, such as lymphocytes. In one embodiment, a cytokine is a chemokine, a molecule whose functions include the direction of cellular trafficking. Specific non-limiting examples of cytokines are interferon gamma (IFN-γ), tumor necrosis factor-alpha (TNF-α), TNF-β, IL-2, IL-4, IL-10, amongst others. Specific non-limiting examples of chemokines are macrophage inflammatory protein (MIP) 1-alpha, MIP1-beta, RANTES (regulated upon activation, normal T cell expressed, and presumably secreted), IL-8. Cytokines, including chemokines, may be encoded by host cells, donor cells, or may be encoded by pathogenic organisms such as viruses.

Differentiation: The process by which cells become more specialized to perform biological functions. Differentiation is a property that is often totally or partially lost by cells that have undergone malignant transformation.

Enhancement (enhancing): An increase in a particular parameter of a cell or organism. In one embodiment, enhancement refers to a 25%, 50%, 100% or greater than 100% increase in a parameter. In one specific, non-limiting example, enhancement of hematopoiesis refers to an increase in a population of the cells of a hematopoietic lineage (e.g. B cells, T cells, macrophages, monocytes, or a hematopoietic intermediate cell), such as a 25%, 50%, or 100% increase in the population of cells or the response of the population of cells.

Fetal liver Kinase-1 (Flk-1): A receptor tyrosine kinase closely related to fins-like tyrosine kinase (flt). Flk-1 has been cloned from mouse cell populations enriched for hematopoietic stem cells and progenitor cells, Human kinase insert domain receptor (KDR) and mouse flk-1 show 85 percent amino acid identity. Mouse flk-1 is expressed selectively in vascular endothelium and has been shown also to function as a vascular endothelial growth factor (VEGF) receptor. It has been shown that flk-1 is expressed abundantly in proliferating endothelial cells of the vascular sprouts and branching vessels of embryonic and early postnatal brain and that its expression is reduced drastically in adult brain where proliferation has ceased. Flk-1 is expressed also in the blood islands in the yolk sac of embryos.

GATA: A family of transcription factors that includes GATA-1, GATA-2, and GATA-3. The GATA factors share extensive homology in their DNA-binding domains and are coexpressed in erythroid cells.

The gene encoding GATA-1 has been identified as cMG1, CL100 or Erf-1. GATA-1 recognizes the general consensus motif WGATAR and is expressed predominantly in erythroid cells. GATA-1 is expressed highly in the megakaryocytic and mast cell lineages, but not in other blood cell lineages or in non-hematopoietic cells. GATA-1 activity is required for normal erythroid development and is induced by treatment of cells with a variety of hematopoietic growth factors. GATA-1 regulates erythroid-expressed genes in maturing erythroblasts, including the expression of the Epo receptor. GATA-2 has been shown to be required for expression of the prepro-protein forms of endothelins gene expression in vascular endothelial cells.

Growth Factor: A “growth factor” is a substance that affects the growth of a cell or organism. In general, growth factors stimulate cell proliferation or maturation when they bind to their receptor (“growth factor receptor”). In one embodiment, growth factors are a complex family of polypeptide hormones or biological factors that are control growth, division and maturation of hematopoietic cells. In another embodiment, growth factors regulate the division and proliferation of cells and influence the growth rate of neoplastic tissue (e.g. cancers). A growth factor can be a naturally occurring factor or a factor synthesized using molecular biology techniques. In one specific, non-limiting example, a growth factor can be used stimulate lymphocyte production or differentiation, and thus can be used following chemotherapy or bone marrow transplantation.

Examples include growth factors, epidermal growth factor, platelet-derived growth factor, fibroblast growth factor. Insulin and somatomedin are also growth factors, the status of nerve growth factor is more uncertain. Perturbation of growth factor production or of the response to growth factor is important in neoplastic transformation.

A growth factor that affects the development (maturation), differentiation, division, or proliferation of hematopoietic cells is a “hematopoietic growth factor.”

A “stem cell growth factor” is a growth factor that affects hematopoietic stem cells. Specific nonlimiting examples of a stem cell growth factor are c-kit ligand (e.g. steel factor, stem cell factor) also FLT-3 ligand and LIF.

Hematopoiesis: The formation and development of blood cells. Hematopoiesis involves the proliferation and differentiation from stem cells. In adult mammals hematopoiesis is known to occur in bone marrow. Hematopoiesis is the production of hematopoietic cells including B Cells, T cells, cells of the monocyte macrophage lineage, and red blood cells.

Immunologically Normal: “Immunologically normal” denotes an individual that displays immune system characteristics typical for the species to which the individual belongs. These characteristics would typically include, among others, functioning B cells and T cells as well as structural cell components, called cell surface antigens, which act as the immunologic signature for a particular organism.

The use of such immunologically normal recipients means that an immunologically normal recipient's immune system, via its B-(humoral response) and T-(cellular response) cells, will identify the cell surface antigens of a foreign cell or an engrafted tissue as foreign. This recognition leads ultimately to an immune response against the cell or tissue, resulting in destruction of the cell or rejection of the graft. An immune response against an allogeneic tissue is known as host-versus-graft rejection.

Immunologically Compromised: An “immunologically compromised recipient” is a subject with a genotypic or a phenotypic immunodeficiency. A genotypically-immunodeficient subject has a genetic defect which result in the inability to generate either humoral or cell-mediated response. A specific, non-limiting example of a genotypically immunodeficient subject is a genotypically immunodeficient mouse, such as a SCID mouse or a bg/nu/xid mice (Andriole et al., J. Immunol. 135:2911, 1985; McCune et al., Science 241:1632, 1988). In one embodiment, a genotypically immunodeficient subject is unable to react against a foreign cell or engrafted allogeneic tissue. A “phenotypically-immunodeficient subject” is a subject which is genetically capable of generating an immune response, yet has been phenotypically altered such that no response is seen. In one specific, non-limiting example, a phenotypically-immunodeficient recipient is irradiated. In another specific, non-limiting example, a phenotypically-immunodeficient subject has been treated with chemotherapy.

Inhibition (inhibiting): An decrease in a particular parameter of a cell or organism. In one embodiment, inhibition refers to a 25%, 50%, 100% or greater than 100% decrease in a parameter. In one specific, non-limiting example, inhibition of hematopoiesis refers to a decrease in a population of the cells of a hematopoietic lineage (e.g. B cells, T cells, macrophages, monocytes, or a hematopoietic intermediate cell), such as a 25%, 50%, or 100% decrease in the population of cells or the response of the population of cells.

Isolated: An “isolated” biological component (such as a nucleic acid molecule, protein or portion of a vascular tissue) has been substantially separated or purified away from other biological components in the cell of the organism in which the component naturally occurs. An “isolated” cell is a cell that has been purified from the other cellular components of a tissue. Cells can be isolated by mechanical and/or enzymatic methods. In one embodiment, an isolated population of cells includes greater than about 95%, or greater than about 99% of the cells of interest. In another embodiment, an isolated population of cells is one in which no other cells of a different phenotype can be detected. In a further embodiment, an isolate population of cells is a population of cells that includes less than about 5%, or less than about 1% of a cells of a different phenotype than the cells of interest.

Nucleic acids and proteins that have been “isolated” include nucleic acids and proteins purified by standard purification methods. The term also embraces nucleic acids and proteins prepared by recombinant expression in a host cell as well as chemically synthesized nucleic acids.

Lineage specific marker: A marker that is expressed by a specific population of cells. In one embodiment, the cells are cells of a blood vessel, other than endothelial cells, such as smooth muscle cells. In another embodiment, the cells are a population of immune cells, such as lymphocytes. In one specific, non-limiting example, the marker is a B cell specific marker, such as B220. In another specific, non-limiting example, the marker is a T cell specific marker, such as CD3, CD4, or CD8. In a further specific, non-limiting example the marker is a macrophage specific marker, such as Mac-1. Cocktails of antibodies that bind lineage specific markers has been produced (see U.S. Pat. No. 5,061,620, herein incorporated by reference). Cells that do express B220, Mac-1, CD3, CD5, NK1.1, CD4, CD8 and CD45 are referred to herein as lin⁻.

Leukocyte: Cells in the blood, also termed “white cells,” that are involved in defending the body against infective organisms and foreign substances. Leukocytes are produced in the bone marrow. There are 5 main types of white blood cell, subdivided between two main groups: polymorphonuclear leukocytes (neutrophils, eosinophils, basophils) and mononuclear leukocytes (monocytes and lymphocytes). When an infection is present, the production of leukocytes increases.

Lymphocytes: A type of white blood cell that is involved in the immune defenses of the body. There are two main types of lymphocytes: B cell and T cells.

Lymphoproliferation: An increase in the production of lymphocytes.

Mammal: This term includes both human and non-human mammals. Similarly, the term “subject” includes both human and veterinary subjects.

Maturation: The process in which an immature cell, such as a precursor cell, changes in form or function to become a functional mature cell, such as a mature T or B cell.

Microvasculature: The portion of the vasculature comprising the finer blood vessels. In one embodiment, the microvasculature is blood vessels having an internal diameter of 100 microns or less.

Monocyte: A large white blood cell in the blood that ingests microbes or other cells and foreign particles. When a monocyte passes out of the bloodstream and enters tissues, it develops into a macrophage.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Similarly, an IRES (internal ribosomal entry site) is operably linked to a coding sequence if it allows entry of a ribosome, and subsequent translation of the coding sequence. Generally, operably linked DNA sequences are contiguous. Expression of two genes encoded by the same plasmid is regarded as operably linked if they are driven by one promoter and an IRES.

Pharmaceutically acceptable carriers: The pharmaceutically acceptable carriers useful in this invention are conventional. Remington's Pharmaceutical Sciences, by E. W. Martin, Mack Publishing Co., Easton, Pa., 15th Edition (1975), describes compositions and formulations suitable for pharmaceutical delivery of the fusion proteins herein disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Polynucleotide: A linear nucleotide sequence, including sequences of greater than 100 nucleotide bases in length.

Polypeptide: Any chain of amino acids, regardless of length or post-translational modification (e.g., glycosylation or phosphorylation).

Progenitor Cell: A “progenitor cell” is a cell that gives rise to progeny in a defined cell lineage. A “hematopoietic progenitor cell” is a cell that gives rise to cells of the hematopoietic lineage. One specific non-limiting example of a hematopoietic progenitor cell is a “common lymphoid progenitor cell,” which is a progenitor cell that gives rise to immature and mature lymphoid cells. Another specific, non-limiting example of a hematopoietic progenitor cells is a “T cell progenitor cell,” which gives rise to immature and mature T cells. Yet another specific, non-limiting example of a progenitor cell is a “stromal progenitor cell,” which is a progenitor cell that gives rise to stromal elements.

Promoter: A promoter is an array of nucleic acid control sequences which direct transcription of a nucleic acid. A promoter includes necessary nucleic acid sequences near the start site of transcription, such as, in the case of a polymerase II type promoter, a TATA element. A promoter also optionally includes distal enhancer or repressor elements which can be located as much as several thousand base pairs from the start site of transcription. The promoter can be a constitutive or an inducible promoter. A specific, non-limiting example of a promoter is the HCMV IE promoter.

Purified: The term “purified” does not require absolute purity; rather, it is intended as a relative term. Thus, for example, a purified cell preparation is one in which the cell referred to is more pure than the cell in its natural environment within a tissue. In one embodiment, a “substantially purified” population of a specific cell type is a composition of cells includes less than about 20%, less than about 15%, or less than about 10% of cell of a different phenotype. Thus, a substantially purified population of cells includes greater than 80%, greater than 85%, or greater than 90% of the cells of interest. In another embodiment, a process that produces purified population of cells is a process that produces a population of cells so that more than 50% of the resulting population is the cell type of interest.

Recombinant: A recombinant nucleic acid is one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, e.g., by genetic engineering techniques. Similarly, a recombinant protein is one encoded for by a recombinant nucleic acid molecule.

Stem cell antigen-1 (Sca-1): A murine cell surface antigen also called also Ly-6A/E. Sca-1 is expressed on immature hematopoietic progenitor cells and stem cells. Antibodies directed against Sca-1 have been used to fractionate early hematopoietic progenitor cells that have the ability to repopulate bone marrow in vivo.

Stem Cell: A “stem cell” is a pluripotent cell that gives rise to progeny in all defined hematolymphoid lineages. In addition, limiting numbers of cells are capable of fully reconstituting a seriously immunocompromised subject in all blood cell types and their progenitors, including the pluripotent hematopoietic stem cell by cell renewal.

Subject: Any subject that has a vascular system and has hematopoietic cells in the wild-type organism. In one embodiment, the subject is a non-human mammalian subject, such as a monkey, mouse, rat, rabbit, pig, goat, sheep or cow. In another embodiment, the subject is a human subject.

Supernatant: The culture medium in which a cell is grown. The culture medium includes material from the cell, including secreted growth factors.

T Cell: A white blood cell critical to the immune response. T cells include, but are not limited to, CD4⁺ T cells and CD8⁺ T cells. A CD4⁺ T lymphocyte is an immune cell that carries a marker on its surface known as “cluster of differentiation 4” (CD4). These cells, also known as helper T cells, help orchestrate the immune response, including antibody responses as well as killer T cell responses. CD8⁺ T cells carry the “cluster of differentiation 8” (CD8) marker. In one embodiment, a CD8 T cells is a cytotoxic T lymphocytes. In another embodiment, a CD8 cell is a suppressor T cell.

Tyrosine kinase that contains immunoglobulin-like loops and EGFhomology domains-2 (TIE-2): A receptor-like tyrosine kinase called also TEK, that is expressed predominantly in the endothelium of actively growing blood vessels. Signaling through the TIE-2 receptor involves binding of SH-PTP2 and GRB-2. By using the yeast two-hybrid system one of the downstream signaling partners of the activated TIE-2 receptor has been identified as Dok-R. The TIE-2 ligand has been identified as Angiopoietin-1. TIE-2 has been shown to be expressed also on hematopoietic progenitor cells and some leukemic blasts. See Huang et al., Oncogene 11(10): 2097-103, 1995.

Therapeutically effective amount of a cell: An amount of a CD31⁺CD34⁺CD45⁻CD105⁺lin⁻ c-kit⁻ cell, that can be determined by various methods, including generating an empirical dose-response curve, predicting potency and efficacy of using modeling, and other methods used in the biological sciences. In general, a therapeutically effective amount of CD31⁺CD34⁺CD45⁻CD105⁺lin⁻ c-kit⁻ cell is an amount sufficient to reconstitute hematopoiesis in an immunocompromised subject. In one embodiment, a therapeutically effective amount of CD31⁺CD34⁺CD45⁻CD105⁺lin⁻ c-kit⁻ cells is more than about 10,000 cells, more than about 20,000 cells, more than about 30,000 cells, or between about 5,000 cells and about 50,000 cells.

The therapeutically effective amount of cells will be dependent on the subject being treated (e.g. the species or size of the subject), the degree that the subject is immunocompromised, and the location of the transplant (e.g. intraperitoneal, kidney capsule, etc). In one embodiment, a therapeutically effective amount of cells is an amount of cells sufficient to increase the number of B cells, T cells, monocytes and/or macrophages in the peripheral blood of a recipient.

Specific assays for determining the therapeutically effective amount of CD31⁺CD34⁺CD45⁻CD105⁺lin⁻ c-kit⁻ cells are provided herein. The methods disclosed in the present invention have equal application in medical and veterinary settings. Therefore, the general term “subject being treated” is understood to include all animals (e.g. humans, apes, dogs, cats, mice, rats, rabbits, sheep, pigs, mice and cows) and reconstitution of hematopoiesis is monitored using the assays described herein. For example, the presence of B cells, T cells, or macrophages following transplantation can be determined.

Transduced and Transformed: A virus or vector “transduces” a cell when it transfers nucleic acid into the cell. A cell is “transformed” by a nucleic acid transduced into the cell when the DNA becomes stably replicated by the cell, either by incorporation of the nucleic acid into the cellular genome, or by episomal replication. As used herein, the term transformation encompasses all techniques by which a nucleic acid molecule might be introduced into such a cell, including transfection with viral vectors, transformation with plasmid vectors, and introduction of naked DNA by electroporation, lipofection, and particle gun acceleration.

Transplantation: The transfer of a tissue or an organ, or cells, from one body or part of the body to another body or part of the body. An “allogeneic transplantation” or a “heterologous transplantation” is transplantation from one individual to another, wherein the individuals have genes at one or more loci that are not identical in sequence in the two individuals. An allogeneic transplantation can occur between two individuals of the same species, who differ genetically, or between individuals of two different species. An “autologous transplantation” is a transplantation of a tissue or cells from one location to another in the same individual, or transplantation of a tissue or cells from one individual to another, wherein the two individuals are genetically identical.

Vascular Tissue: Tissue consisting of, or containing, vessels as an essential part of a structure. Vascular tissue operates by means of, or is made up of an arrangement of, vessels. Vascular tissue includes the arteries, veins, capillaries, lacteals, microvasculature, etc. In one embodiment, vascular tissue includes a highly vascularized organ (e.g. the lung). In another embodiment, vascular tissue is a blood vessel, or a portion thereof. Cells isolated from a vascular tissue are a population of cells isolated from the remaining components of the tissue. One specific, non-limiting example of cells from a vascular tissue are endothelial cells isolated from vascular tissue, such as a blood vessel.

Vector: In one embodiment a nucleic acid molecule as introduced into a host cell, thereby producing a transformed host cell. A vector may include nucleic acid sequences that permit it to replicate in the host cell, such as an origin of replication. A vector may also include one or more selectable marker genes and other genetic elements known in the art. In one embodiment the term “vector” includes viral vectors, such as adenoviruses, adeno-associated viruses, vaccinia, and retroviral vectors. In one embodiment the term vector includes bacterial vectors.

Von Willebrand factor: A plasma protein central to the blood coagulation system. Deficiencies in (vWF) result in von Willebrand Disease (vWD), the most common inherited bleeding disorder in humans. Recently the gene for von Willebrand factor has been cloned using endothelial cell DNA. All types of endothelial cells synthesize vWF.

Isolated Cells that Enhance Hematopoietic Stem Cell Survival

An isolated cell that promotes hematopoietic stem cell survival is disclosed wherein the cell expresses CD31, CD34 and CD105 (CD31⁺CD34⁺CD105⁺ cells). These cells are lin⁻ as they do not express B220, Mac-1, CD3, CD5, CD4, CD8, CD45, or NK1.1. These cells also do not express c-kit (ckit⁻) or CD45 (CD45⁻). Thus, disclosed herein are CD31⁺CD34⁺CD45⁺CD105⁺lin⁻c-kit⁻cells. In one embodiment, the cell expresses von Willebrand factor, Flk-t or Tie-2. In an additional embodiment, the cell does not express β-H1 or mB-1. In a second embodiment, the cell does not express one or more additional hematopoietic cell lineage specific markers. In one embodiment, the cell is a human cell. In another embodiment, the cell is a murine cell, and expresses Sca-1. Thus, in this embodiment, the cell is a CD31⁺CD34⁺CD45⁻CD105⁺⁻lin⁻c-kit-Sca-1⁺ cell.

In one embodiment, a composition is provided that includes a substantially purified population of cells that are characterized as expressing CD31, CD34 and CD105, and not expressing CD45, c-kit or a hematopoietic cell lineage marker (CD31⁺CD34⁺CD45⁻CD105⁺⁻lin⁻c-kit⁻), e.g. human cells that are CD31⁺CD34⁺CD45⁻CD105⁺lin⁻c-kit⁻). In several embodiments, the composition comprises fewer than about 20%, about 10%, about 5%, or about 1% of lineage committed cells. In several other embodiments the composition comprises greater than about 80%, greater than about 90%, greater than about 95%, or greater than about 99% CD31⁺CD34⁺CD45⁻CD105⁺inc-kit⁻ cells.

The cells can be isolated from any source of endothelial cells, including adult and fetal mammalian tissues. In one specific-non-limiting example, the cells are from a blood vessel. In another specific, non-limiting example, the cells are from the bone marrow. In yet another specific, non-limiting example the cells are isolated from endothelial cells of the lung, brain, or kidney, although the cells can be isolated from the microvasculature of any organ. The cells are mammalian cells. In one specific, non-limiting example the cells are murine cells. In another specific, non-limiting example the cells are human cells.

In one embodiment, a method is provided for isolating CD31⁺CD34⁺CD45⁻CD105⁺lin⁻ c-kit⁻ cells. In one specific, non-limiting example, the resulting population of cells includes CD31⁺CD34⁺CD45⁻CD105⁺lin⁻ c-kit⁻ cells as greater than 50% of the population, greater than 80% of the population, greater than 90% of the population, greater than 95% of the population, or greater than 90% of the population. In order to purify CD31⁺CD34⁺CD45⁻CD105⁺lin⁻ c-kit⁻ cells, the method can be performed a single time on a sample, or can be performed more than one time in succession.

In one embodiment, suspension of cells including endothelial cells is produced, and antibodies that specifically bind CD31 is reacted with the cells in suspension. Methods of determining the presence or absence of a cell surface marker, such as CD31, are well known in the art. Typically, labeled antibodies specifically directed to the marker are used to identify the cell population. The antibodies can be conjugated to other compounds including, but not limited to, enzymes, magnetic beads, colloidal magnetic beads, haptens, fluorochromes, metal compounds, radioactive compounds or drugs. The enzymes that can be conjugated to the antibodies include, but are not limited to, alkaline phosphatase, peroxidase, urease and β-galactosidase. The fluorochromes that can be conjugated to the antibodies include, but are not limited to, fluorescein isothiocyanate, tetramethylrhodamine isothiocyanate, phycoerythrin, allophycocyanins and Texas Red. For additional fluorochromes that can be conjugated to antibodies see Haugland, R. P., Molecular Probes: Handbook of Fluorescent Probes and Research Chemicals (1992-1994). The metal compounds that can be conjugated to the antibodies include, but are not limited to, ferritin, colloidal gold, and particularly, colloidal superparamagnetic beads. The haptens that can be conjugated to the antibodies include, but are not limited to, biotin, digoxigenin, oxazalone, and nitrophenol. The radioactive compounds that can be conjugated or incorporated into the antibodies are known to the art, and include but are not limited to technetium 99m (⁹⁹Tc), ¹²⁵I and amino acids comprising any radionuclides, including, but not limited to, ¹⁴C, ³H and ³⁵S.

Fluorescence activated cell sorting (FACS) can be used to sort cells that express CD31, by contact the cells with an appropriately labeled antibody. In one embodiment, additional antibodies and FACS sorting can further be used to produce substantially purified populations of CD31⁺CD34⁺CD45⁻CD105⁺lin⁻ c-kit⁻ cells, or to purify cells that express Sca-1.

A FACS employs a plurality of color channels, low angle and obtuse light-scattering detection channels, and impedance channels, among other more sophisticated levels of detection, to separate or sort cells. Any FACS technique may be employed as long as it is not detrimental to the viability of the desired cells. (For exemplary methods of FACS see U.S. Pat. No. 5,061,620, herein incorporated by reference). Similarly, FACS can be used to substantially purify lin⁻ cells that express CD31, CD34, and CD105, but do not express Sca-1 or CD5.

However, other techniques of differing efficacy may be employed to purify and isolate desired populations of cells. The separation techniques employed should maximize the retention of viability of the fraction of the cells to be collected. The particular technique employed will, of course, depend upon the efficiency of separation, cytotoxicity of the method, the ease and speed of separation, and what equipment and/or technical skill is required.

Separation procedures may include magnetic separation, using antibody-coated magnetic beads, affinity chromatography, cytotoxic agents, either joined to a monoclonal antibody or used in conjunction with complement, and “panning,” which utilizes a monoclonal antibody attached to a solid matrix, or another convenient technique. Antibodies attached to magnetic beads and other solid matrices, such as agarose beads, polystyrene beads, hollow fiber membranes and plastic petri dishes, allow for direct separation. Cells that are bound by the antibody can be removed from the cell suspension by simply physically separating the solid support from the cell suspension. The exact conditions and duration of incubation of the cells with the solid phase-linked antibodies will depend upon several factors specific to the system employed. The selection of appropriate conditions, however, is well within the skill in the art.

The unbound cells then can be eluted or washed away with physiologic buffer after sufficient time has been allowed for the cells expressing a marker of interest (e.g. CD31) to bind to the solid-phase linked antibodies. The bound cells are then separated from the solid phase by any appropriate method, depending mainly upon the nature of the solid phase and the antibody employed.

Antibodies may be conjugated to biotin, which then can be removed with avidin or streptavidin bound to a support, or fluorochromes, which can be used with a fluorescence activated cell sorter (FACS), to enable cell separation (see above).

The CD31⁺CD34⁺CD45⁻CD105⁺lin⁻c-kit⁻ cells initially may be separated from other cells by the cell-surface expression of CD31. In one specific, non-limiting example, CD31⁺ cells are positively selected by magnetic bead separation, wherein magnetic beads are coated with CD31⁻ reactive monoclonal antibody. The CD31⁺ cells then are then removed from the magnetic beads.

Release of the CD31⁺ cells from the magnetic beads can effected by culture release or other methods. Purity of the isolated CD31⁺ cells is then checked with a FACSCAN.RTM. flow cytometer (Becton Dickinson, San Jose, Calif.), for example, if so desired. In one embodiment, further purification steps are performed, such as FACS sorting the population of cells released from the magnetic beads.

In one embodiment, magnetic bead separation is used to first separate a population of cells that do not express more than one lineage specific markers, for example, B220, CD4, CD8, CD3, CD45, CD5, Mac-1, and/or NK1.1. In addition, panning can be used to separate cells that do not express one or more lineage specific markers, or that to separate cells CD31, CD34, and CD105 (for panning methods see Small et al., J Immunol Methods 3; 167(1-2):103-7, 1994, herein incorporated by reference).

In one embodiment, CD31⁺CD34⁺CD45⁻CD105⁺lin⁻ c-kit⁻ cell secretes growth factors or cytokines. In one embodiment, the cell secretes known cytokine or growth factor. In another embodiment, the cell secretes a novel cytokine or growth factor. Thus, included in this disclosure is a novel cytokine or growth factor that supports stem cell growth, survival or differentiation that is secreted by CD31⁺CD34⁺CD45⁻CD105⁺lin⁻ c-kit⁻ cells. In another embodiment, the CD31⁺CD34⁺CD45⁻CD105⁺lin⁻ c-kit⁻ cells promote the survival, differentiation, or proliferation of stem cells by cell-to-cell contact. Thus, methods are disclosed herein which utilize cell-to-cell contact to promote the growth, survival and/or differentiation of hematopoietic stem cells.

CD31⁺CD25⁺CD45⁻CD105⁺lin⁻c-kit⁻ cells can be transduced using standard procedures known in molecular biology in order to introduce a nucleic acid molecule of interest into the cell. In one embodiment, the nucleic acid molecule encodes a polypeptide. The polypeptide encoded by the nucleic acid molecule can be from the same species as the cells (homologous), or can be from a different species (heterologous). For example, a nucleic acid molecule can be utilized that supplements or replaces deficient production of a peptide by the tissue of the host wherein such deficiency is a cause of the symptoms of a particular disorder. In this case, the cells act as a source of the peptide. In one specific, non-limiting example the polypeptide is a cytokine or a growth factor for hematopoietic cells.

In one embodiment, the nucleic acid molecule of interest encodes a polypeptide involved in growth regulation or neoplastic transformation of CD31⁺CD34⁺CD45⁻CD105⁺lin c-kit⁻. Specific, non-limiting examples of nucleic acids sequences of interest are SV40 Tag, p53, myc, src, and bcl-2. In another embodiment, the nucleic acid sequence of interest encodes an enzyme. Specific, non-limiting examples of enzymes are proteins involved in the conversion of a pro-drug to a drug, or growth factors that promote the expansion, differentiation, or survival of hematopoietic cells. In yet another embodiment, the nucleic acid sequence of interest encodes a transcriptional regulator.

In one embodiment, the nucleic acid sequence of interest is operably linked to a regulatory element, such as a transcriptional and/or translational regulatory element. Regulatory elements include elements such as a promoter, an initiation codon, a stop codon, mRNA stability regulatory elements, and a polyadenylation signal. A promoter can be a constitutive promoter or an inducible promoter. Specific non-limiting examples of promoters include the immunoglobulin promoter, or a T cell specific promoter, and promoters including TET-responsive element for inducible expression of transgene. In another embodiment, the nucleic acid sequence of interest is inserted into a vector, such as an expression vector. Procedures for preparing expression vectors are known to those of skill in the art and can be found in Sambrook et al., Molecular Cloning: A Laboratory Manual, 2nd Ed., Cold Spring Harbor Press, Cold Spring Harbor, N.Y. (1989). Expression of the nucleic acid of interest occurs when the expression vector is introduced into an appropriate host cell. The vector can be a viral vector, such as an adenoviral or retroviral vector.

Retroviral vectors of use are produced recombinantly by procedures already taught in the art. For example, WO 94/29438 describes the construction of retroviral packaging plasmids and packaging cell lines. The techniques used to construct vectors, and transfect and infect cells are widely practiced in the art. Examples of retroviral vectors are those derived from murine, avian or primate retroviruses. Retroviral vectors based on the Moloney (Mo) murine leukemia virus (MuLV) are the most commonly used because of the availability of retroviral variants that efficiently infect human cells. Other suitable vectors include those based on the Gibbon Ape Leukemia Virus (GALV) or HIV.

In producing retroviral vector constructs derived from the Moloney murine leukemia virus (MoMLV), in most cases, the viral gag, pol and env sequences are removed from the virus, creating room for insertion of foreign DNA sequences. Genes encoded by the foreign DNA are usually expressed under the control of the strong viral promoter in the LTR. Such a construct can be packed into viral particles efficiently if the gag, pol and env functions are provided in trans by a packaging cell line. Thus, when the vector construct is introduced into the packaging cell, the gag-pol and env proteins produced by the cell, assemble with the vector RNA to produce infectious virions that are secreted into the culture medium. The virus thus produced can infect and integrate into the DNA of the target cell, but does not produce infectious viral particles since it is lacking essential packaging sequences. Most of the packaging cell lines currently in use have been transfected with separate plasmids, each containing one of the necessary coding sequences, so that multiple recombination events are necessary before a replication competent virus can be produced. Alternatively, the packaging cell line harbors an integrated provirus. The provirus has been crippled so that, although it produces all the proteins required to assemble infectious viruses, its own RNA cannot be packaged into virus. Instead, RNA produced from the recombinant virus is packaged. The virus stock released from the packaging cells thus contains only recombinant virus. The range of host cells that may be infected by a retrovirus or retroviral vector is determined by the viral envelope protein. The recombinant virus can be used to infect virtually any other cell type recognized by the env protein provided by the packaging cell, resulting in the integration of the viral genome in the transduced cell and the stable production of the foreign gene product. In general, murine ecotropic env of MoMLV allows infection of rodent cells, whereas amphotropic env allows infection of rodent, avian and some primate cells, including human cells. Amphotropic packaging cell lines for use with MoMLV systems are known in the art and commercially available and include, but are not limited to, PA 12 and PA317. Miller et al., Mol. Cell. Biol. 5:431-437, 1985; Miller et al., Mol. Cell. Biol. 6:2895-2902, 1986; and Danos et al., Proc. Natl. Acad. Sci. USA 85:6460-6464, 1988. Xenotropic vector systems exist which also allow infection of human cells (U.S. Pat. No. 5,638,928, which is herein incorporated by reference).

In yet another specific, non-limiting example, a nucleic acid sequence can be introduced to decrease rejection. For example, the immunogenicity of a cell may be suppressed by deleting genes that produce proteins that are recognized as “foreign” by the host (a knock-out), or by introducing genes which produce proteins, such as proteins that are native to the host and recognized as “self” proteins by the host immune system.

Thus in one embodiment, the cells can be transfected with a nucleic acid molecule designed to functionally delete or “knock-out” a gene of interest. In this method, the nucleic acid molecule of interest is a nucleic acid molecule that undergoes homologous recombination and is inserted into the genome of the cell. Methods for producing “knock-outs” in ES cells are known to one of skill in the art (e.g. see U.S. Pat. No. 5,939,598). CD31⁺CD34⁺CD105⁺lin⁻c-kit⁻ cells can be isolated from a knock out animal and used in the methods disclosed herein.

According to this example, cells are cultured in vitro as described herein and an exogenous nucleic acid is introduced into the cells by any method known to one of skill in the art, for example, by transfection or electroporation. The transfected cultured cells can then be studied in vitro or can be administered to a subject (see below). Methods for the introduction of nucleic acid sequences into stem cells are known in the art (e.g., see U.S. Pat. No. 6,110,743; U.S. Pat. No. 5,164,397; U.S. Pat. No. 5,928,639, all of which are incorporated by reference herein).

### Methods of Use

Methods are disclosed herein for reconstituting hematopoiesis in a subject. The methods include administering to the subject a therapeutically effective amount of cells expressing CD31, CD34 and CD105, and not expressing CD45, c-kit or a hematopoietic cell lineage marker to reconstitute hematopoiesis. Without being bound by theory, in one embodiment hematopoiesis is reconstituted by the expression of a cytokine by the CD31⁺CD34⁺CD45⁻CD105⁺lin⁻c-kit⁻ cells. The secreted cytokine affects the survival, proliferation, or differentiation of the subjects' hematopoietic stem cells, thereby reconstituting hematopoiesis in the subject. Without being bound by theory, in another embodiment hematopoiesis is reconstituted by cells to cell contact of the CD31⁺CD34⁺CD45⁻CD105⁺lin⁻c-kit⁻ cells with the cells of the recipient, such as with hematopoietic stem cells of the recipient.

In several embodiments, following transplantation of the CD31⁺CD34⁺CD45⁻CD105⁺lin⁻c-kit⁻, the subject's hemoglobin level is increased, platelet count is increased, and/or white blood cell count is increased (including, but not limited to an increase in T cells or B cells). Hematopoiesis is reconstituted when at least one of the following parameters increases: platelet count, T cell count, B cell count, antibody production, hemoglobin level, number of natural killer cells, or number of macrophages.

The composition may be administered intravenously to a subject requiring a bone marrow transplant in an amount sufficient to reconstitute the patient's hematopoietic and immune systems. The composition may be supplemented with hematopoietic stem cells and other lineage-uncommitted cells.

Precise, effective quantities can be readily determined by those who are skilled in the art and will depend, of course, upon the exact condition being treated by the particular therapy being employed.

A survey of published reports indicates that the number of CFU-GM infused for autologous bone marrow reconstitution in human patients can be relied on as an indicator of the potential for successful hematopoietic reconstitution (Spitzer et al., Blood 55(2): 317-323, 1980; Douay et al., Exp. Hematol. 14:358-365, 1986).

In one embodiment, greater than 2×10⁶/kg of CD31⁺CD34⁺CD45⁻CD105⁺lin⁻c-kit⁻ cells is administered to the subject. In another embodiment, 2×10⁶/kg-5×10⁶/kg CD31⁺CD34⁺CD45⁻CD105⁺lin⁻c-kit⁻ cells are administered. Without being bound by theory, these doses of CD31⁺CD34⁺CD45⁻CD105⁺lin⁻c-kit⁻ cells are applicable in both autologous and allogeneic settings (Sezer et al., J. Clin. One. 18:3319-3320, 2000; Mavroudis et al., Blood 88:3223-3229, 1996). Accordingly, it is anticipated that the administration of compositions of the present invention comprising an equivalent or greater number of CD31⁺CD34⁺CD45⁻CD105⁺lin⁻c-kit⁻ cells, either alone or in combination with stem/progenitor cells, should result in the successful reconstitution of a human hematopoietic system. In a further embodiment, other agents, such as growth factors or cytokines are administered in conjunction with CD31⁺CD34⁺CD45-CD105⁺lin⁻c-kit cells. These agents can be administered before, after, or simultaneously with the CD31⁺CD34⁺CD45⁻CD105⁺lin⁻c-kit⁻ cells. One or multiple doses can be administered.

In another embodiment, CD31⁺CD34⁺CD45⁻CD105⁺lin⁻c-kit⁻ cells are co-cultured with hematopoietic stem cells in vitro to increase the survival or differentiation of the hematopoietic stem cells. For example, hematopoietic stem cells are isolated and propagated by growing in conditioned medium from stromal cells, such as those that can be obtained from bone marrow or liver associated with the secretion of factors, or in medium comprising cell surface factors supporting the proliferation of stem cells (e.g. see U.S. Pat. No. 5,164,397 and U.S. Pat. No. 5,087,570, which are herein incorporated by reference). The CD31⁺CD34⁺CD45⁻CD105⁺lin⁻c-kit⁻ cells can be added to these cultures to increase the survival or differentiation of the hematopoietic stem cells. In another embodiment, CD31⁺CD34⁺CD45⁻CD105⁺lin⁻c-kit⁻ cells are isolated and cultured. The cell culture supernatant is collected, and cytokines are purified from the supernatent. These cytokines are of use in promoting the growth, survival and/or differentiation of hematopoietic stem cells.

Unless otherwise explained, all technical and scientific terms used herein have the same meaning as commonly understood by one of ordinary skill in the art to which this invention belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present invention, suitable methods and materials are described below. The term “comprises” means “includes.” All publications, patent applications, patents, and other references mentioned herein are incorporated by reference in their entirety. In case of conflict, the present specification, including explanations of terms, will control. In addition, the materials, methods, and examples are illustrative only and not intended to be limiting.

The invention is illustrated by the following non-limiting Examples.

### EXAMPLES

### Example 1

### Material and Methods

Animals. Mice were purchased from the Jackson Laboratory (Bar Harbor, Me.) or bred and maintained in an appropriate animal care facility. C57BL/6 Ly5.2 mice aged 8-12 weeks were used as donors and age matched C57B1/6 Ly5.1 mice or C57B1/6 Ly5.1×C57B1/6 Ly5.2 mice were used as recipients. Recipient mice were maintained on acidified water (PH 2.2) prior to transplantation.

CD31 cell preparation. Brain and lung tissue was collected from donor mice washed with modified Hank's Balanced Salt Solution (HBSS contains 5% FCS and 10 mM HEPES buffer), digested with 0.5% collagenase in DMEM containing 1% Pen/Strep, 10 mM HEPES and 3% BSA at 37° C. for 1 hour. Digested tissue was passed through 70 μm filter to remove tissue debris. Brain tissue was resuspended over a BSA cushion (25% BSA in Buffer A) and centrifuged at 2500 rpm for 20 minutes to remove lipid containing cells. Cell pellets from both brain and lung tissue preparations were washed with HBSS prior to antibody staining.

Cell surface marker analysis and cell sorting. Cell suspensions prepared from the brain and lung were washed and incubated with monoclonal antibodies to CD31 and Sca-1(Pharmingen San Diego Calif.). A Becton-Dickinson FACS Vantage® cell sorter was used to sort CD31 positive cells from both brain and lung cell preparations. Typically 5-10% of the total cell isolated from the brain and lungs expressed CD31 and these cells were predominately low side scatter with low-intermediate forward scatter. Residual erythrocytes, dead cells and debris were excluded from the sorted populations by light scattering gating and PI (propidium iodide) staining. Re-analysis of the sorted cell populations typically demonstrated a purity of ≧95%. Detailed phenotypic analysis of CD31 positive cells was performed using a FACSCaliber® cytometer (Becton-Dickinson, San Jose Calif.). In each experiment, between 5,000-10,000 CD31 positive cells were evaluated for the co-expression of Sca-1, CD34, CD105, VE Cadherin, c-kit, CD45, Ter 19, Mac-1, Gr-1, B220, CD3, CD5, NK1.1. The expression of an individual cell surface marker on ≧1.0% of the CD31⁺ cells was considered positive.

Molecular phenotyping of brain and lung derived CD31 positive cells. Total RNA was isolated from 2000-5000 of sorted CD31 positive cells purified from brain and lung and from normal, unfractionated adult bone marrow cells using Qiagen one-step RT-PCR kit (Qiagen, Valencia, Calif.). The total reaction volume was 50 μl, and reactions proceeded at 94° C., 1 minute, 58° C., 1 minute, and 72° C., 1 minute, for a total of 30 cycles. The PCR products were electrophoresed on the 1% agarose gel and visualized with ethidium bromide. Expression of all genes was analyzed in at least 2 independent experiments utilizing sorted populations of CD31+ cells form at least 2 different cell sorting procedures. PCR primer sequences used are as follows:

| | |
|---|---|
| βH1(βchain of hemoglobin) | |
| 5′-AGT CCC CAT GGA GTC AAA GA | (SEQ ID NO: 1) |
| 5′-CTC AAG GAG ACC TTT GCT CA | (SEQ ID NO: 2) |
| c-kit, | |
| 5′-TGT CTC TCC AGT TTC CCT GC | (SEQ ID NO: 3) |
| 5═-TTC AGG GAC TCA TGG GCT CA | (SEQ ID NO: 4) |
| GATA-1, | |
| 5′-ATG CCT GTA ATC CCA GCA CT | (SEQ ID NO: 5) |
| 5′-TCA TGG TGG TAG CTG GTA GC | (SEQ ID NO: 6) |
| HPRT, | |
| 5′CACAGGACTAGAACACCTGC | (SEQ ID NO: 7) |
| 5′-GCTGGTGAAAAGGACCTCT | (SEQ ID NO: 8) |
| Tie-2, | |
| 5′-GGATGGCAATCGAATCACTG | (SEQ ID NO: 9) |
| 5′-TCTGCTCTAGGCTGCTTCTT | (SEQ ID NO: 10) |
| Flk-1, | |
| 5′-TGAGCCAAGTGTTAAGTGTGG | (SEQ ID NO: 11) |
| 5′-GAGCAAGCTGCATCATTTCC | (SEQ ID NO: 12) |
| CD-31, | |
| 5′-AGGGGACCAGCTGCACATTAGG | (SEQ ID NO: 13) |
| 5′AGGCCGCTTCTCTTGACCACTT | (SEQ ID NO: 14) |
| Mb-1, | |
| 5′-GCC AGG GGG TCT AGA AGC | (SEQ ID NO: 15) |
| 5′-TCA CTT GGC ACC CAG TAC AA | (SEQ ID NO: 16) |
| vWF, | |
| 5′-CTC AGA GCT TCG GCG CAT CAC CAG | (SEQ ID NO: 17) |
| 5′-GAC AAA CAC CAC ATC CAG AAC CAT | (SEQ ID NO: 18) |
| PU-1, | |
| 5″- AACCACTTCACAGAGCTGCA | (SEQ ID NO: 19) |
| 5′- CAAGCCATCAGCTTCTCCAT | (SEQ ID NO: 20) |
| GATA-2, | |
| 5′-GACTATGGCAGCAGTCTCTTCC | (SEQ ID NO: 21) |
| 5′-GGTGGTTGTCGTCTGACAATT | (SEQ ID NO: 22) |

Radioprotection Assay. Recipient mice were irradiated with a bone marrow lethal dose of gamma radiation (1200 cGy, delivered in 2 fractions of 600cGy, given 3 hours apart using a J. L. Shepherd Co. Cesium irradiator). Sorted populations of CD31+cells from donor brain or lung were resuspended in 200 μl of HBSS at doses of 30,000, 10,000 and 1000 per recipient and injected i.v. into the retro-orbital plexus under methoxyflurane anesthesia. Control mice were injected with HBSS only. Irradiated mice were maintained on aqueous antibiotics (Polymyxin B sulfate at 10⁶ unit/liter and neomycin sulfate at 1.1 g/liter) and monitored daily for survival for 60 days.

Hematopoietic Reconstitution. Peripheral blood (PB) was obtained by retroorbital puncture. Aliquots of 200 μl were collected in Microtainer tubes with EDTA (Becton Dickinson) and analyzed for total leukocytes, serum hemoglobin, and platelet counts (Antech Diagnostics, Portland, Oreg.). For the determination of donor derived hematopoiesis, PB was collected in 200 μl Hanks balanced salt solution/10 mM HEPES/3% fetal calf serum (Modified HBSS) with heparin at 50 units/ml. Recipient tissue analyzed for the presence of donor derived cells as follows. Total nucleated cells were prepared by sedimenting erythrocytes in 2% Dextran (T-500) followed by hypotonic lysis. Donor and recipient mice were congenic at the Ly-5 locus (CD45) allowing the quantitation of percentage of percentage of donor and host derived cells by flow cytometry. Nucleated cells were incubated with donor (Ly5.2) and lineage specific antibodies as described previously. Cell pellets were washed and incubated with Ly5.2-FITC and Ly5.2-PE in combination with lineage specific markers for T cells (CD3-APC), B cells (B220-APC) or myelomonocytic cells (Mac-1-APC and Gr-1-APC) (Pharmingen, San Diego, Calif.). The co-expression of these cell surface antigens on donor (Ly5.2) and host hematopoietic cells (Ly5.1) was determined using a FACSCaliber® cytometer. Dead cells were excluded using scatter gates and propidium iodide. Up to 50 thousand events were analyzed to provide a sensitivity of ≧0.2% donor derived cells.

Secondary Transplantation. Donor BM was obtained from primary transplant animals that had been radioprotected by 10,000 CD31⁺ cells from brain six months previously. Secondary recipients were lethally irradiated to a dose of 1100 cGy and 2×10⁶ BM cells in a total volume of 200 ul HBSS were injected into each secondary transplant recipient. These secondary recipients were maintained on antibiotic water and survival was monitored daily for 60 days. The peripheral blood of recipient mice was analyzed by flow cytometry for the presence of multilineage donor cell at 4 weeks post-transplant and at 24 weeks.

### Example 2

### Phenotype of Isolated CD31⁺ Cells

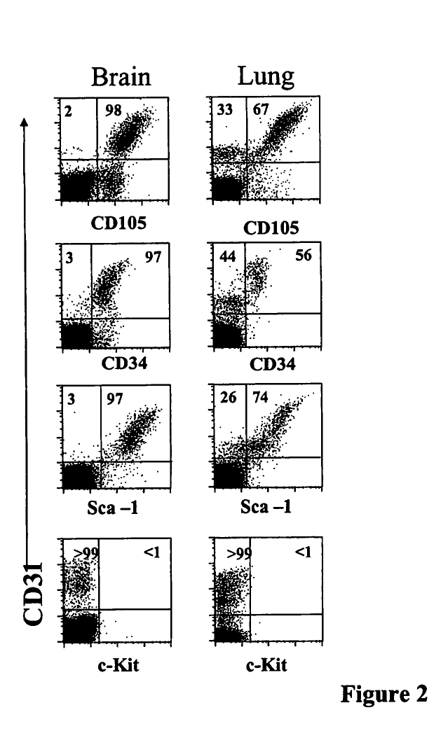

CD31⁺ cells were isolated from lungs and brains of C57BL/6 mice as described above (see Example 1). The expression of markers on the isolated endothelial (CD31⁺) cells was then assessed using specific antibodies and a FACSCaliber® cytometer (see Example 1). The FACS profiles for CD31⁺ cells expressing CD105, CD34, c-kit and sca-1 is shown in FIG. 2.

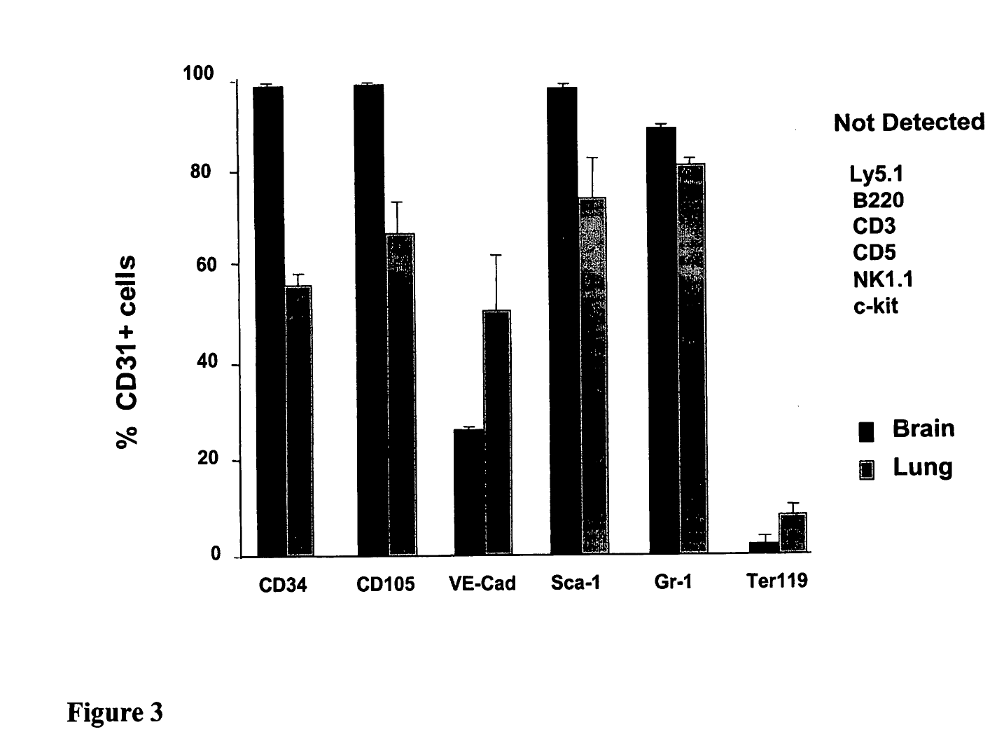

Less than 1% of the CD31⁺ cells isolated from the brain or the lung expressed c-kit. However, co-expression of all of the other markers with CD31 was observed. For example, 98% of the CD31⁺ cells isolated from the brain expressed CD105, while only 67% of the CD31⁺ cells isolated from the lung expressed CD105. Similarly, 97% of the CD31⁺ cells isolated from the brain expressed CD34, while only 56% of the CD31⁺ cells isolated from the lung expressed CD34. In addition, 97% of the CD31⁺ cells isolated from the brain co-expressed Sca-1, while only 74% of the CD31⁺ cells isolated from the lung expressed Sca-1 (see FIG. 2). It should be noted that 30% of the brain derived CD31⁺ cells expressed VE-Cad, and grater than 90% of the brain derived CD31⁺ cells expressed Gr-1, while less than 5% of the brain derived CD31⁺ cells expressed Ter19 (see FIG. 3). Fifty-five percent of lung derived CD31⁺ cells expressed VE-Cad, and greater than 85% of the lung derived CD31⁺ cells expressed Gr-1, while less than 15% of the lung derived CD31⁺ cells expressed Ter19 (see FIG. 3).

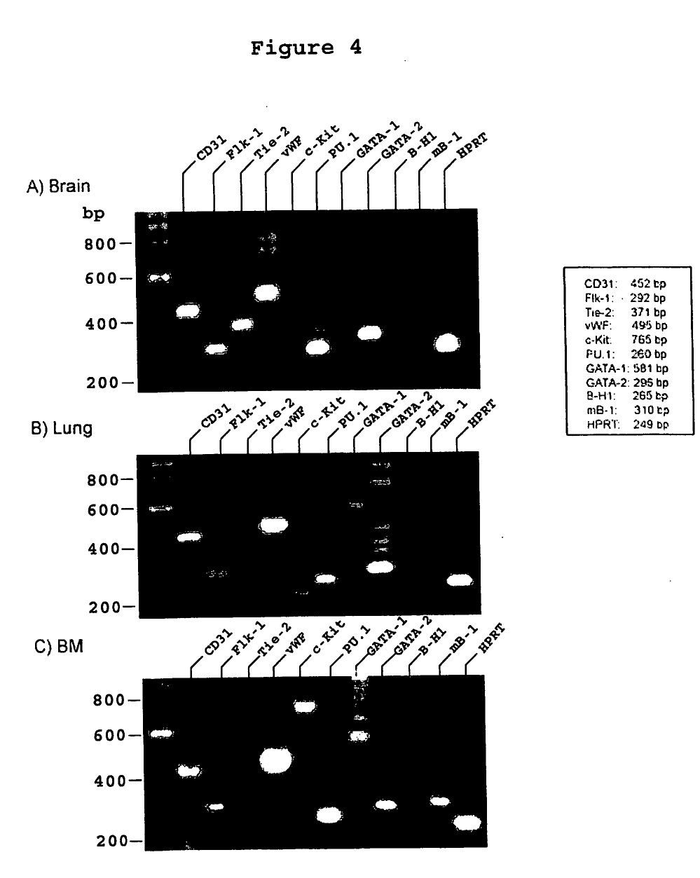

To asses the molecular profile of the CD31⁺ cells, PCR analyses were performed to examine the transcript of mRNAs encoding Flk-1, Tie-2, von Willebrand factor (vWF), c-kit, PU.1, GATA-1, GATA-2, β-H1), mB-1, and HPRT (FIG. 4). Probes for CD31 were included as a positive control. As shown in FIG. 4A CD31⁺ cells isolated from the brain expressed Flk-1, Tie-2, von Willebrand factor, PU.1, GATA-2 and HPRT, but did not express c-kit, GATA-1, β-H1 or mB-1. As shown in FIG. 4B, CD31⁺ cells isolated from the lung also expressed Flk-1, Tie-2, von Willebrand factor, PU.1 (a stem cell marker), GATA-2 and HPRT (hypoxanthine-guanine phosphoribosyltransferase), but did not express c-kit, GATA-1, β-H1 or mB-1. FIG. 4C, shows the results obtained with unfractionated bone marrow cells, which were included as a positive control.

### Example 3

### CD31⁺CD34⁺CD45⁻CD105⁺lin⁻c-kit⁻ Cells Rescue Lethally Irradiated Mice

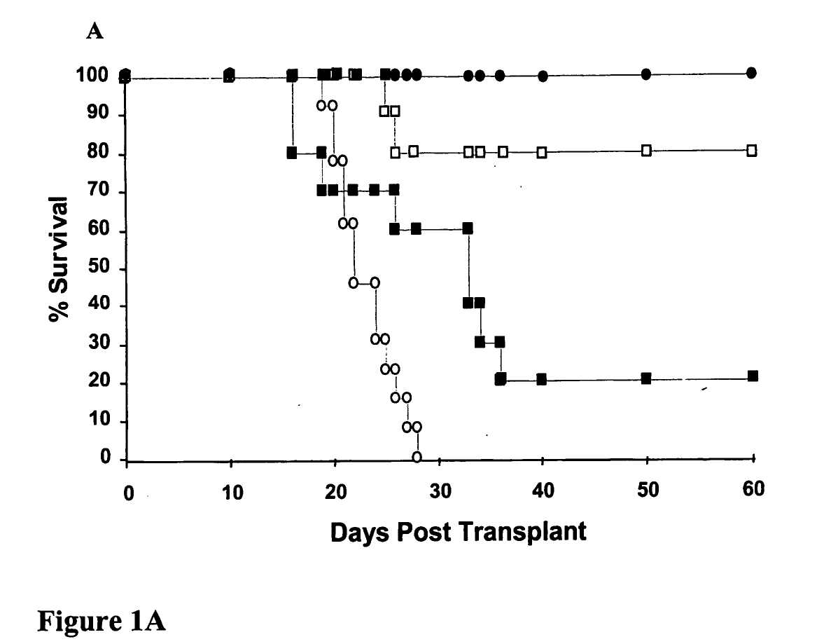

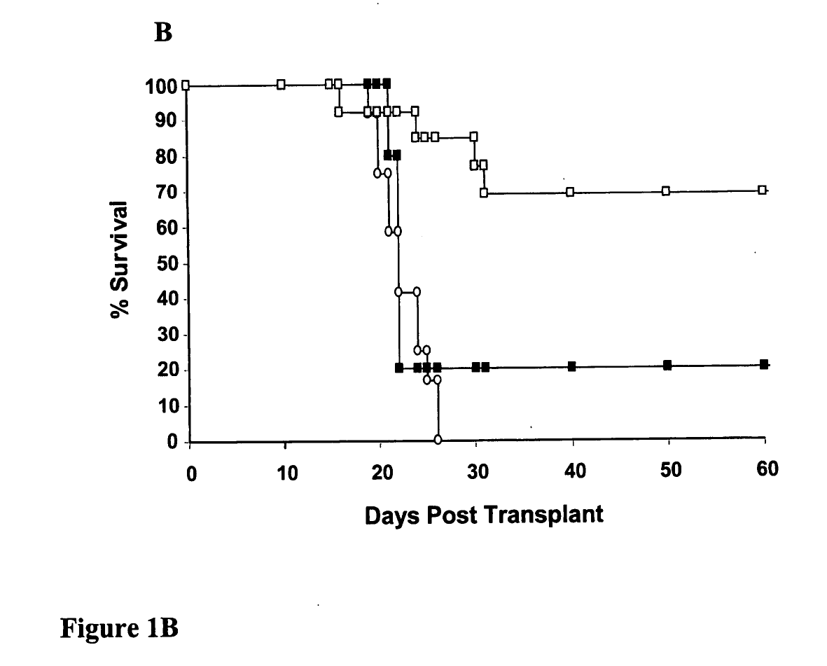

Transplantation studies demonstrated a dose dependent rescue of lethally irradiated mice (FIG. 1). Brain and lung tissue was collected from donor mice washed with modified Hank's Balanced Salt Solution, and CD31⁺ cells were prepared, as described above (see Example 1). Recipient mice were irradiated with a bone marrow lethal dose of gamma radiation (1200 cGy, delivered in 2 fractions of 600 cGy, given 3 hours apart). Sorted populations of CD31⁺ cells from donor brain or lung were resuspended in 200 μl of HBSS at doses of 30,000, 10,000 and 1000 per recipient and injected i.v. Survival was monitored over time, and the experiment was terminated 60 days after transplantation.

As shown in FIG. 1A, all mice survived following intravenous injection of 30,000 brain-derived CD31⁺ cells, while 80% survived when 10,000 brain-derived CD31⁺ cells were administered. Only 20% of the animals survived the monitoring period when 1,000 brain derived CD31⁺ cells were administered. The results obtained with lung-derived CD31⁺ cells are shown in FIG. 1B. All of the animals survived following intravenous injection of 30,000 lung-derived CD31⁺ cells, while 70% percent of the animals survived after the injection of 30,000 CD31⁺ cells. Twenty percent of the animals survived when 1,000 CD31⁺ cells were injected. For comparison, 2×10⁵ unfractionated bone marrow cells were required to save a lethally irradiated mouse.

A peripheral blood analysis of the radioprotected recipients was also performed. Six months after irradiation, the peripheral blood of mice rescued with either CD31⁺ cells brain derived cells, or with bone marrow cells, was evaluated for circulating cells, hemoglobin concentration and platelets. The results for each group is shown in Table 1 below (values are shown as mean+/−SEM):

TABLE 1

| Input | Nucleated | Lymphs/ | Neutrophils/ | Monocytes/ | Eosin/ | Hgb | Plateletes × |
|---|---|---|---|---|---|---|---|
| Cells | Cells/μl | μl | μl | μl | μl | g/dl | 10³/μl |
| BM | 7750 + 751 | 6946 +/− 718 | 715 +/− 87.1 | 35 +/− 20 | 52.5 +/− 17.5 | 13.2 +/− 0.2 | 903.0 +/− 34.8 |
| CD31 | 3428 +/− 570 | 2134 +/− 521 | 761 +/− 84.7 | 305 +/− 61.9 | 67.6 +/− 27.2 | 13.1 +/− 0.3 | 1003.4 +/− 71.9 |

Peripheral blood of recipient mice analyzed six months after transplantation showed normal hemoglobin levels, platelet counts, and neutrophil counts. However, a 2-3 fold decrease in the absolute numbers of circulating CD3+ and B220+ lymphocytes was observed.

### Example 4

### Lethally Irradiated Mice Rescued with CD31⁺CD34⁺CD45⁻CD105⁺in⁻c-kit⁻ Cells are Reconstituted with Recipient Cells

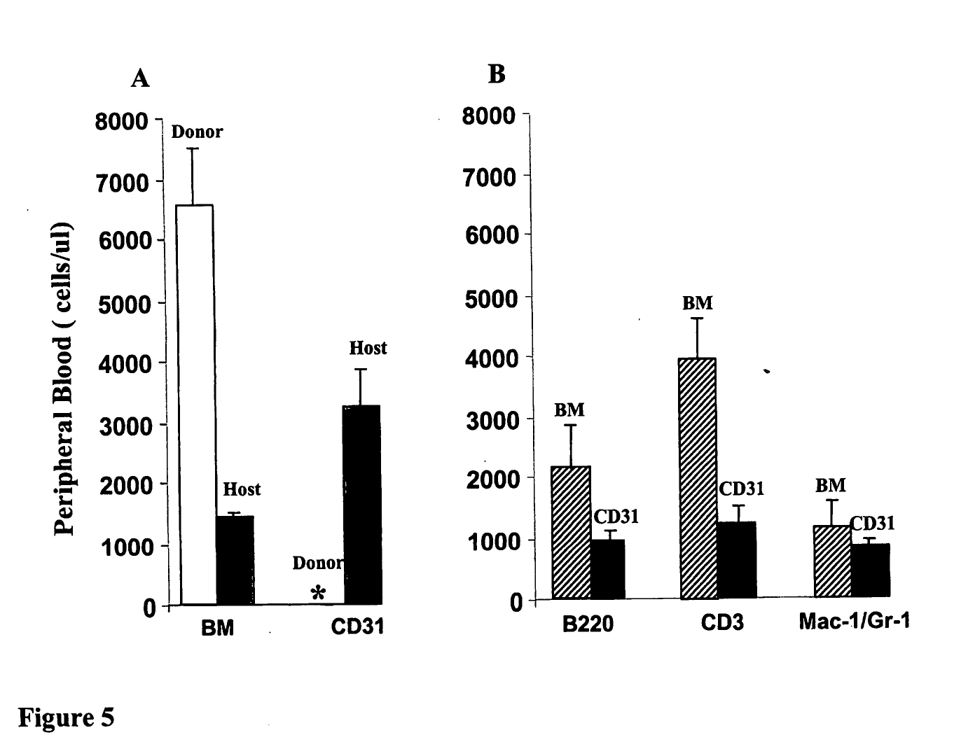

In order to determine the origin of the hematopoietic cells in the recipient animals, donor and recipient mice utilized that were congenic at the Ly-5 locus (CD45) allowing the quantitation of percentage of percentage of donor and host derived cells by flow cytometry. Six months after transplantation of CD31⁺ cells isolated from Ly5.2 donors into lethally irradiated Ly5.1 recipients, nucleated cells were isolated and incubated with donor (Ly5.2) and lineage specific antibodies as described above in Example 1. Cell pellets were washed and incubated with Ly5.2-FITC and Ly5.2-PE in combination with lineage specific markers for T cells (CD3-APC), B cells (B220-APC) or myelomonocytic cells (Mac-1-APC and Gr-1-APC) (Pharmingen, San Diego, Calif.). The co-expression of these cell surface antigens on donor (Ly5.2) and host hematopoietic cells (Ly5.1) was determined using a FACSCaliber® cytometer. The mean percentage of donor derived and host derived cells in the peripheral blood is shown in FIG. 5. When peripheral blood was analyzed, only host derived (Ly5.1 cells) were detected (sensitivity 0.2%). The results indicate that donor CD31⁺ cells from the brain and lung have the capacity to rescue host hematopoiesis from the effects of lethal irradiation, and demonstrate that these CD31⁺ cells affect the proliferation, survival, and/or differentiation of host hematopoietic stem cells.

### Example 5

### Secondary Transplantation

As described above, at 60 days following multilineage hematopoietic reconstituion in congenic mice, there were no donor derived cells detected in recipients peripheral blood. At eight months after primary transplantation, mice were scarified; both peripheral blood and bone marrow were collected for flow analysis to search for donor cells. There were still no donor derived cells seen in recipients peripheral blood. To demonstrate that host derived stem cells (HSC) were radioprotected, whole bone marrow was harvested, 2×10⁶ of total bone marrow cells were transplanted into lethally irradiated recipient mice.

Briefly, bone marrow was obtained from brain CD31⁺ cells radioprotected recipients eight months after the primary transplantation. Secondary recipients were lethally irradiated to a dose of 1200 cGy (600 cGy two times, 3 hrs apart) and 2×10⁶ BM cells in a total volume of 200 ul Hank's Balanced Saline Solution (HBSS) were injected into each secondary transplanted recipient mice. These secondary recipients were maintained on antibiotic water and survival was monitored daily for at least 50 days. The peripheral blood of recipient mice was analyzed by flow cytometry for the presences of multilineage donor derived cells at 6 weeks after secondary transplantation.

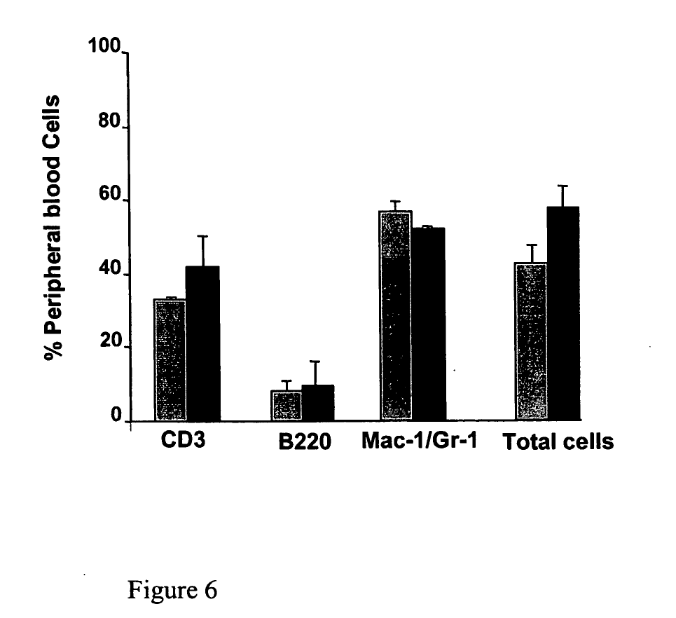

Peripheral blood analysis from the recipients analyzed six weeks after transplantation showed primary host derived cells in irradiated recipients' bone marrow could repopulate blood cells in the secondary recipient. To evaluate multilineage hematopoiesis, the peripheral blood was analyzed for the presence of primary recipient derived B-cells (B220), T-cells (CD3) and Myeloid (Mac-1/Gr-1) markers. B220⁺ cells, CD3⁺ cells, and Mac-1/Gr-1⁺ cells were present (FIG. 6). This formally demonstrates radioprotection of hematopoietic stem cells.

### Example 6

### Isolation of Cytokines from CD31⁺ Cells

In order to determine if cytokines are secreted by CD31⁺ cells, the CD31⁺ cells are isolated and placed in implantable Millipore filters and transplanted subcutaneously or intraperitoneally. In this system, cytokines can diffuse out but the CD31⁺ cells remain in filter chamber devices. Transplantation of CD31⁺ cells placed in Millipore filters rescues lethally irradiated animals, demonstrating expression of one or more cytokines that affect the donor hematopoietic stem cells.

Gene expression profiles in CD31⁺ cells and CD31⁺ cells implanted into irradiated recipients are compared. Novel gene products which are responsible for host radioprotection are isolated.

### Example 7

### Method of Isolating Human CD31⁺CD34⁺CD45⁻CD105⁺lin⁻c-kif⁻ Cells

CD31⁺ cells are isolated from human peripheral blood or bone marrow using magnetic beads coated with antibody to CD31 (Dynal, Lake Success; see Hewett et al., Eur J. Cell Biol. 62:451-454, 1993). Fluorescence activated cell sorting (FACS) analysis is used to determine that the cells express CD31. Briefly, single-donor human peripheral blood is obtained with a 20-gauge intravenous catheter. The first 3 ml is discarded, and the leukocyte fraction is obtained by Ficoll density gradient centrifugation. The cells are plated on plastic tissue culture dishes for one hour to avoid contamination by differentiated adhesive cells. Similarly, CD31⁺ cells can be obtained from aspirated bone marrow. It should be noted that fluorescence activated cell sorting can also be utilized to isolate CD31⁺ cells from the peripheral blood, bone marrow, or cell suspensions from human biopsies.

### Example 8

### Protocol for Reconstituting Hematopoiesis in Bone Marrow Transplant Recipients

PBSC or bone marrow cells are collected in the recovery phase of consolidation chemotherapy with a continuous-flow blood cell separator (Fenwal CS3000, Baxter Healthcare Corp., IL or Cobe Spectra Apheresis System, Cobe Laboratories Inc., Lakewood, Colo.), with or without recombinant human granulocyte colony-stimulating factor (G-CSF, filgrastim) or macrophage-CSF (M-CSF, Mirimostim) for mobilization of PBSC. The use of hematopoietic growth factors depended on the decision of the institution. The administration of G-CSF begins from absolute granulocyte count (AGC)<0.5×10⁹/l at a doses of 200 μg/m²/day intravenously. M-CSF is used from 5 days after completion of chemotherapy at a dose of 800×10⁶ units/m²/day intravenously. The apheresis is done in the recovery phase of chemotherapy when platelet (>100×10⁹/l) counts were increasing rapidly. In one embodiment, hematopoietic stem cells and/or CD 31⁺ cells are isolated prior to cyropreservation. The collected cells are then cryopreserved using the uncontrolled hydroxyethyl starch (HES)-dimethyl sulfoxide (DMSO) method as previously described. Frozen cells are stored in the liquid phase of liquid nitrogen or in an electric freezer (Sanyo Electric, Tokyo) at −135° C.

Cytoreductive Regimens

The pretransplant cytoreductive regimen is used at the discretion of the participating institute. For example, a combination of busulfan (Bu, 16 mg/kg), VP-16 (50 mg/kg) and Ara-C (3 g/m² b.i.d. for 4 days), is administered as defined in the CCLSG protocol. In another example, MCNU (250 mg/m² on day −8 and 200 mg/m² on day −3), VP-16 (200 mg/m²) and Ara-C (2.0 g/m²) (each b.i.d. on days −7 through −4) and cyclophosphamide (50 mg/kg on days −2 and −1) are administered. Alternatively, TBI (Ara-C 2.5 g/m² b.i.d. for 5 days+VP-16 60 mg/kg+10 Gy TBI) is administered. If the subjects are children, for all of the protocols, children younger than 5 years received all drugs adjusted to mg/kg of body weight (1 m²=30 kg). In older children, the doses are determined based on the body surface calculated by the true or ideal body weight, whichever was less.

Transplant Procedure

Thirty-six hours after completion of the cytoreductive regimen, cryopreserved cells were thawed at 37° C. and promptly infused into the patient through a central venous catheter without additional post-thaw washing (day 0). Several transplant procedures are known in the art. However, in one embodiment, the patients receive prophylactic acyclovir 15 mg/kg/day orally or intravenously starting on the first day of the conditioning regimen. Broad-spectrum antibiotics and amphotericin B are given only when clinically indicated. Blood products are given as needed to maintain a hemoglobin level of 8 g/dl and a platelet count of 20×10⁹/l. In all cases, platelets were collected from a single donor by apheresis. In selected patients, recombinant G-CSF is administered after PBSCT once daily as a 60 min infusion for 14 days. PRT was graded according to the standard World Health Organization (WHO) system. In one group of patients, about 2×10⁶/kg of CD31⁺CD34⁺CD45-CD105⁺lin⁻c-kit⁻ cells is administered to the subject. In another embodiment, about 5×10⁶/kg CD31⁺CD34⁺CD45⁻CD105⁺lin⁻c-kit⁻ cells are administered. In another group of patients, hematopoietic stem cells are administered in conjunction with the CD31⁺CD34⁺CD45⁻CD105⁺lin⁻c-kit⁻ cells. In a forth group of patients, autologous bone marrow cells are administered in conjunction with CD31⁺CD34³⁰ CD45⁻CD105⁺lin⁻c-kit⁻ cells (see Sezer et al., J. Clin. One. 18:3319-3320, 2000; Mavroudis et al., Blood 88:3223-3229, 1996).

Statistical Analysis

Patients are followed up at several time points over five years post-transplant. For example, the patients can be examined monthly over the first year, bimonthly over the second year, and ever six months following. One of skill in the art can readily determine appropriate periods for follow up. The following definitions of survival were used: overall survival is the time from transplant to death; disease-free survival (DFS) is the time of transplant to relapse or death; relapse-free survival (RFS) is the time from transplant to relapse (censoring at death in CR). Overall survival, DFS and RFS are determined by the Kaplan-Meier method. Different actuarial curves are compared using the log-rank test. Possible prognostic factors which may affect RFS included age and WBC count at diagnosis, FAB classification, the number and type of transplanted cells, the timing of apheresis and PBSCT, use of CSF for mobilization and type of conditioning regimen. Variables that potentially affected RFS are assessed in a multivariate analysis by the Cox proportional hazard model using a stepwise regression procedure. Correlation between the type of cells transplanted and the numbers of mononuclear cells (MNC), CD34+ cells or colony forming unit-granulocyte/macrophage (CFU-GM) collected by apheresis are computed using the Mann-Whitney test. A univariate analysis of factors affected hematopoietic recovery is performed with the use of Kaplan-Meier analysis and the log-rank test.

It will be apparent that the precise details of the methods or compositions described may be varied or modified without departing from the spirit of the described invention. We claim all such modifications and variations that fall within the scope and spirit of the claims below.

1. A composition comprising lin⁻ cells that are characterized as expressing CD31, CD34 and CD105, and not expressing c-kit, wherein the composition comprises fewer than 20% of lineage committed cells.

2. The composition of claim 1, wherein the cells express at least one of von Willebrand factor, Flk-1, and Tie-2.

3. The composition of claim 1, wherein the cells do not express B-H1 and mB-1.

4. The composition of claim 1, wherein the composition comprises fewer than 10% of lineage committed cells.

5. The composition of claim 1, wherein the composition comprises greater than about 80% CD31⁺34⁺CD45⁻CD105⁺ c-kit⁻lin⁻cells.

6. The composition of claim 1, wherein the composition comprises greater than about 90% CD31⁺34⁺CD45⁻CD105⁺ c-kit⁻lin⁻cells.

7. The composition of claim 1, wherein the composition comprises greater than about 95% CD31⁺34⁺CD45⁻CD105⁺ c-kit⁻lin⁻ cells.

8. The composition of claim 1, wherein the composition comprises greater than about 99% CD31⁺34⁺CD45⁻CD105⁺c-kit⁻lin⁻ cells.

9. The composition of claim 5, wherein the CD31⁺34⁺CD45⁻CD105⁺ c-kit⁻lin⁻ cells are microvasculature cells.

10. A composition comprising substantially purified CD31⁺34⁺CD45⁻CD105⁺ c-kit⁻lin⁻ cells.

11. The composition of claim 10, wherein the cells express Sca-1.

12. The composition of claim 10, wherein the cells are murine cells.

13. The composition of claim 10, wherein the cells are human cells.

14. The composition of claim 10, wherein the CD31⁺34⁺CD45⁻CD105⁺ c-kit⁻lin⁻ cells are microvasculature cells.

15. A method of preparing a composition comprising a purified population of cells, wherein greater than 50% of the cells are CD31⁺CD34⁺CD45⁻CD105⁺c-kit⁻lin⁻ cells, comprising
contacting cells of the vasculature with an antibody that specifically binds CD31; and
separating cells that bind the antibody from the vasculature,
thereby isolating a population of cells that are CD31⁺CD34⁺CD45⁻CD105⁺c-kit⁻lin⁻.

16. The method of claim 15, wherein the cells are murine cells.

17. The method of claim 15, wherein the cells are human cells.

18. The method of claim 15, wherein the microvasculature is the microvasculature of the brain or the lung.

19. The method of claim 15, wherein the CD31⁺CD34⁺CD45⁻CD105⁺c-kit⁻lin⁻ cells are microvasculature cells.

20. The method of claim 15, wherein the purified population comprises greater than about 80% CD31⁺CD34⁺CD45⁻CD105⁺c-kit⁻lin⁻ cells.

21. The method of claim 15, wherein the purified population comprises greater than about 90% CD31⁺CD34⁺CD45⁻CD105⁺c-kit⁻lin⁻ cells.

22. The method of claim 15, wherein the purified population comprises greater than about 95% CD31⁺CD34⁺CD45⁻CD105⁺c-kit⁻lin⁻ cells.

23. The method of claim 15, wherein the purified population comprises greater than about 99% CD31⁺CD34⁺CD45⁻CD105⁺c-kit⁻lin⁻ cells.

24. A method of reconstituting hematopoiesis in a subject, comprising administering to the subject a therapeutically effective amount of a composition comprising CD31⁺CD34⁺CD45⁻CD105⁺c-kit⁻lin⁻ cells, thereby reconstituting hematopoiesis.

25. The method of claim 24, wherein the composition comprises autologous cells.

26. The method of claim 24, wherein the composition comprises heterologous cells.

27. The method of claim 24, wherein the subject is a recipient of radiation therapy.

28. The method of claim 24, wherein the subject is a recipient of chemotherapy.

29. The method of claim 24, wherein reconstituting hematopoiesis comprises increasing hemoglobin level.

30. The method of claim 24, wherein reconstituting hematopoiesis comprises increasing platelet count.

31. The method of claim 24, wherein reconstituting hematopoiesis comprises increasing white blood cell count.

32. The method of claim 24, wherein reconstituting hematopoiesis comprises increasing T cell count.

33. The method of claim 24, wherein reconstituting hematopoiesis comprises increasing B cell count.

34. A method of promoting the proliferation or differentiation of a hematopoietic stem cell in a subject, comprising administering to the subject a therapeutically effective amount of the composition of claim 1, thereby promoting the proliferation or survival of the hematopoietic stem cell.

35. The method of claim 34, wherein the hematopoietic stem cell is autologous.

36. The method of claim 34, wherein the hematopoietic stem cell is heterologous.

37. The method of claim 34, wherein the subject is a human subject.

38. The method of claim 24, wherein the subject is exposed to radiation.

39. A pharmaceutical composition comprising a therapeutically effective amount of the composition of claim 1 in a pharmaceutically acceptable medium.

40. A kit for reconstituting hematopoiesis, comprising a container comprising the composition of claim 1 and instructions for administering the composition of claim 1.

41. An isolated cell that promotes hematopoietic stem cell survival, wherein the cell expresses CD31, CD34 and CD105, but does not express c-kit or a hematopoietic lineage specific marker.

42. The isolated cell of claim 41, wherein the cell expresses von Willebrand factor, Flk-t or Tie-2.

43. The isolated cell of claim 41, wherein the cell does not express B-H1 or mB-1.

44. The isolated cell of claim 41, wherein the hematopoietic lineage specific marker is B220, Mac-1, CD3, CD5, NK1.1, CD4, CD8 and CD45.

45. An isolated lin⁻CD31⁺CD34⁺CD45⁻CD105⁺c-kit⁻Sca-1⁺ cell.

46. The isolated cells of claim 45, wherein the cell is a cell of the mircovasculature.

47. A method for promoting proliferation or differentiation of a hematopoietic stem comprising co-culturing the hematopoietic stem cell with a CD31⁺CD34⁺CD45⁻CD105⁺lin⁻ c-kit⁻ cell.

48. A cell isolated by the method of claim 15.
